(19) United States
(12) Patent Application Publication
(10) Pub. No.: US 2026/0237497 A1
(43) Pub. Date: Aug. 13, 2026

(54) System and Method for Real-Time, AI-Optimized Pharmacy Inventory Query and Fulfillment Visibility Without Persistent Data Storage

(71) Applicant: Hayden Sklarow, Chantilly, VA (US)

(72) Inventor: Hayden Sklarow, Chantilly, VA (US)

(21) Appl. No.: 19/250,251

(22) Filed: Jun. 26, 2025

Related U.S. Application Data
(63) Continuation-in-part of application No. 19/052,144, filed on Feb. 12, 2025.

(51) Int. Cl. G16H 40/20; G06F 9/451; G06F 16/2455; G16H 10/60

(52) U.S. Cl. CPC G16H 40/20; G06F 9/451; G06F 16/2455; G16H 10/60

(57) ABSTRACT

A system and method are disclosed for real-time, non-persistent querying of pharmacy inventory availability. The system enables healthcare providers to initiate inventory lookup requests via a stateless application programming interface (API), which routes the request to one or more pharmacy management systems (PMSs). Returned availability data is normalized into a unified schema and presented through a comparative interface without persisting inventory data beyond the transaction. An artificial intelligence (AI) module ranks pharmacies based on weighted criteria including stock level, proximity, insurance compatibility, and historical fulfillment performance. The system further includes a fulfillment feedback mechanism for receiving and displaying real-time status updates from pharmacies regarding prescription outcomes. Interoperability with heterogeneous PMSs is achieved through adapter modules and protocol converters. The invention enhances medication access and prescription efficiency without replicating or storing protected pharmacy inventory data, thereby addressing data privacy and integration constraints in healthcare environments.

### BACKGROUND OF THE INVENTION

### Field of Invention

The present invention relates generally to systems and methods for accessing and communicating medication inventory data. More particularly, the invention pertains to real-time, query-driven technologies for retrieving pharmacy inventory availability without persistent data storage, and to the dynamic presentation of such data to healthcare providers and clinical platforms via application programming interfaces (APIs) and user interfaces. The invention further encompasses integration with heterogeneous pharmacy management systems and the application of artificial intelligence (AI) to optimize pharmacy selection, inventory ranking, and prescription fulfillment efficiency.

### Brief Summary of the Invention

The present invention relates to a system and method for performing real-time, query-based retrieval of medication inventory data from pharmacy systems without storing such data persistently. The system enables healthcare providers to initiate availability checks via an API, which transmits requests to one or more pharmacies and returns current inventory information. The invention also includes optional artificial intelligence functionality to rank and recommend pharmacies based on factors such as proximity, availability, and historical fulfillment performance. Integration is supported across diverse pharmacy systems, including those without standardized APIs, through a normalization layer. In some embodiments of the present invention, patents can also report and view availability via a mobile app.

### BRIEF DESCRIPTION OF THE FIGURES

FIG. 1 illustrates the real-time pharmacy inventory and fulfillment workflow, comprising sequential stages including query initialization (FIG. 1.101), API-based routing (FIG. 1.103), orchestration and dispatch to multiple pharmacies (FIG. 1.105), data normalization (FIG. 1.107), AI-driven ranking (FIG. 1.109), real-time UI rendering (FIG. 1.111), and fulfillment status feedback processing (FIG. 1.113).

### DETAILED DESCRIPTION

The present invention is directed to a non-persistent, real-time medication inventory access system configured to facilitate dynamic, on-demand inventory queries across distributed pharmacy systems. At its core, the architecture is comprised of a stateless application programming interface (API) layer, one or more query orchestration modules, and a secure pass-through infrastructure designed to execute and return medication availability requests without storing or caching inventory data beyond the active transaction lifecycle. The system architecture is designed to comply with prevailing data protection regulations and privacy constraints by obviating the need for long-term or persistent storage of sensitive inventory data, thereby mitigating institutional hesitancy associated with external data replication.

The architecture begins with a request initiation point, typically instantiated by a prescribing clinician or a clinical decision support system via a user interface, such as a web application, mobile interface, or embedded module within an electronic health record (EHR) environment. Upon receiving a medication-specific query, the request is transmitted through the RealTimeRx (RTRx) API gateway. This API layer comprises authentication, authorization, and routing modules which serve to validate the requesting party, enforce access controls, and direct the query to the appropriate downstream endpoints.

Once authenticated, the API delegates the request to a query orchestration module which dynamically constructs a protocol-specific payload for each targeted pharmacy system. The system architecture supports both single-pharmacy and multi-pharmacy request modes. In the multi-pharmacy mode, the orchestration module concurrently issues outbound requests to multiple pharmacy endpoints, leveraging parallel execution threads and connection pooling to optimize query latency and system throughput. Importantly, the system does not retain inventory responses beyond the duration of the query transaction. Returned data is immediately formatted, transmitted to the requester, and then programmatically purged from memory in accordance with strict data ephemerality rules defined within the system's stateless design paradigm.

The invention employs a highly modular service-oriented architecture (SOA), wherein each functional component—such as query normalization, protocol translation, API routing, and session management—is abstracted into discrete microservices. This facilitates independent scalability, fault isolation, and deployment flexibility across heterogeneous infrastructure environments, including containerized platforms and cloud-native orchestration layers such as Kubernetes. Each microservice communicates via encrypted inter-service channels, using RESTful or gRPC-based APIs, with TLS encryption and rotating authentication tokens governed by a centralized identity management system.

In one embodiment, the API gateway includes a policy enforcement engine configured to inspect incoming requests for compliance with predefined operational parameters, including rate limiting, scope constraints, and query content verification. Additionally, the architecture supports real-time logging and auditability via asynchronous event streams, which emit metadata associated with each query (e.g., timestamp, query source, target pharmacy ID) to an external observability layer without exposing or retaining the substantive inventory content. This design ensures full traceability and monitoring capabilities while preserving compliance with security and confidentiality requirements.

Furthermore, the system is engineered for extensibility to accommodate emerging standards in pharmacy data interoperability, such as HL7 FHIR-based medication dispensing resources and NCPDP SCRIPT transactions. However, in recognition of industry fragmentation, the system is capable of functioning in environments where such standards are not implemented, relying instead on bespoke adapters or protocol converters to interact with legacy pharmacy systems.

The architectural framework of the present invention facilitates low-latency, real-time access to distributed pharmacy inventory systems without requiring persistent data storage. By leveraging a stateless, modular, and standards-agnostic design, the invention enables secure, compliant, and scalable delivery of medication availability data to authorized requestors, thereby enhancing clinical decision-making while respecting the data sovereignty and operational boundaries of participating pharmacies.

The present invention further comprises a robust multi-pharmacy integration framework configured to interface with a plurality of heterogeneous pharmacy management systems (PMSs) that vary in technical maturity, data formats, and communication protocols. The interoperability layer enables the centralized system to dynamically retrieve real-time medication availability information across disparate pharmacy infrastructure landscapes, including but not limited to chain pharmacy platforms, independent pharmacies, hospital-based dispensaries, and mail-order fulfillment centers. The interoperability module serves as the technical and semantic bridge between the RTRx API layer and the various downstream pharmacy systems, normalizing inbound and outbound data flows to conform to a unified schema.

Central to this layer is the adapter abstraction engine, which employs a plug-in architecture to support connectivity with non-standardized pharmacy environments. Each adapter module is customized to interface with a particular PMS, taking into account the proprietary APIs, file-based interfaces, or messaging protocols implemented by the target system. These adapter modules are responsible for transforming normalized query requests into PMS-native formats, dispatching them to the designated system endpoints, and converting the returned inventory payloads back into a standardized representation for consumption by the upstream services. The use of canonical data modeling within the normalization layer ensures consistency in the semantic interpretation of medication availability, regardless of system origin.

The invention accommodates both pull-based and push-compatible configurations, although the preferred embodiment operates on a stateless, pull-based architecture to maintain real-time accuracy and data minimization. In pull mode, the system transmits discrete, ephemeral queries to pharmacy systems only upon receiving a user-initiated request, thereby eliminating the need for synchronized data replication or periodic polling mechanisms. The system includes built-in logic for handling latency, timeouts, and error states, with retry strategies and fallbacks engineered to maximize data completeness while avoiding undue load on pharmacy endpoints.

In embodiments where PMSs lack public-facing APIs or network endpoints, the system employs middleware agents installed within the pharmacy's local network. These agents act as secure intermediaries that expose a local interface to the PMS, translate API requests to internal system calls, and return inventory data to the central system through a secure outbound connection. Each agent communicates with the central RTRx platform using encrypted channels and mutual authentication protocols, thereby ensuring data integrity and preventing unauthorized access. The agents can be configured to operate in sandbox or production modes and may include built-in logging and diagnostic capabilities to support ongoing technical support and compliance auditing.

To enable scalability and rapid onboarding of new pharmacy partners, the interoperability layer incorporates a configuration-driven interface mapping system. Pharmacy-specific transformation rules, field mappings, and data validation parameters are defined in declarative configuration files, allowing new systems to be integrated without modifying core code. Additionally, the system supports a certification protocol whereby pharmacy endpoints must undergo a validation sequence to confirm data quality, response format conformance, and uptime reliability prior to being marked as an active data source.

Importantly, the invention includes logic to reconcile discrepancies across pharmacy data sources, such as mismatched drug identifiers, variant units of measure, and conflicting stock statuses. This is achieved through a pharmaceutical normalization engine that utilizes crosswalks for National Drug Codes (NDCs), generic-equivalent mapping tables, and configurable business logic to standardize data across inventory feeds. Such functionality is essential for producing meaningful comparative results across pharmacies and for delivering coherent data to the end-user interface layer.

The multi-pharmacy integration and interoperability framework of the present invention enables real-time access to medication inventory across technically dissimilar and operationally fragmented pharmacy environments. By abstracting the complexity of PMS-specific interfaces and unifying inventory data within a normalized schema, the system delivers seamless, scalable, and secure connectivity that forms the foundation of a responsive, real-time medication visibility network.

The present invention further comprises a comprehensive user interface (UI) delivery subsystem configured to present dynamically retrieved medication inventory data to healthcare providers and authorized users through a variety of digital access points. The system supports delivery across multiple modalities, including web-based portals, native or responsive mobile applications, standalone clinical dashboards, and embedded modules within third-party platforms such as electronic health record (EHR) systems or pharmacy benefit management (PBM) interfaces. Each interface is designed to render real-time inventory results in a human-readable, contextually enriched, and decision-supportive format, thereby enhancing clinical workflows without requiring users to navigate multiple external systems.

Upon completion of a query request through the RTRx API and successful retrieval of inventory data from one or more pharmacy endpoints, the system initiates a presentation pipeline. This pipeline is responsible for formatting the returned data into structured JSON or XML objects, which are then interpreted by the front-end rendering engine associated with the user interface. In the preferred embodiment, the UI leverages reactive front-end frameworks such as React, Angular, or Vue, enabling real-time updates to be reflected within the user session without requiring page reloads or asynchronous polling. This reactive architecture is essential for delivering a seamless and responsive user experience, particularly in clinical environments where speed and reliability are paramount.

The interface includes a comparative inventory visualization component, which aggregates inventory data from multiple pharmacy sources and presents it in a side-by-side or rank-ordered format. Display attributes may include medication name, available quantity, last update timestamp, pharmacy location, affiliation network (e.g., in-network or preferred), estimated fill time, and stock confidence indicators. The system supports filtering and sorting mechanisms based on one or more user-defined or system-inferred criteria, including but not limited to patient ZIP code, distance from the patient or provider, preferred pharmacy networks, and inventory sufficiency thresholds.

To reduce cognitive load and enhance interpretability, the system may employ visual cues such as color-coded status indicators (e.g., green for in-stock, yellow for low-stock, red for out-of-stock), map overlays to depict geospatial pharmacy distribution, and expandable detail panels for each pharmacy listing. Furthermore, the interface includes context-sensitive tooltips, validation messages, and data provenance indicators to ensure transparency and informed decision-making. Inventory data is rendered in near real-time and is refreshed automatically upon re-query or user request, thereby preserving the real-time integrity of the system.

The system also provides an EHR-compatible delivery layer, wherein UI components are exposed as embeddable modules using industry-standard integration mechanisms such as SMART on FHIR, HL7 widgets, or secure iframe-based APIs. These modules allow clinical users to access inventory data directly within their prescribing workflow without leaving the EHR interface. In embodiments where native integration is not feasible, the system provides secure, single-sign-on (SSO) links or redirection endpoints that pre-populate patient and prescription context into the RTRx interface, thereby minimizing data entry duplication and optimizing user experience.

To support cross-platform consistency and accessibility, the system incorporates responsive design principles and conforms to applicable accessibility standards, including the Web Content Accessibility Guidelines (WCAG). Mobile applications may include biometric authentication, offline query caching (limited to non-inventory data), and device-specific features such as geolocation-based pharmacy sorting. All front-end interfaces communicate with the back-end services via secure, authenticated API calls and implement encryption-in-transit protocols such as HTTPS with TLS 1.2 or higher.

The interface layer also serves as the point of origin for user analytics, capturing metadata associated with query behavior, pharmacy selection patterns, and interaction heatmaps. These data are anonymized and analyzed separately to improve system performance and inform the AI optimization engine. Importantly, the interface logic is stateless with respect to inventory content, and no inventory data is cached or stored client-side beyond the scope of the active session, in compliance with the system's overall data minimization philosophy.

The user interface delivery and comparative inventory visualization subsystem of the present invention enables the secure, real-time, and context-aware presentation of medication availability data to authorized users. By combining interoperability with intelligent front-end design, the system facilitates rapid decision-making, enhances prescription accuracy, and reduces barriers to medication access across diverse clinical environments.

The present invention further encompasses an artificial intelligence (AI)-driven optimization subsystem configured to enhance the utility, accuracy, and efficiency of pharmacy selection in the context of real-time inventory availability. This subsystem operates as an intelligent augmentation layer within the broader RTRx platform, leveraging machine learning (ML) models and predictive algorithms to analyze a variety of input signals and generate ranked pharmacy recommendations that maximize the likelihood of successful and timely prescription fulfillment.

At the core of the AI subsystem is a model orchestration engine that integrates supervised, unsupervised, and reinforcement learning techniques. In the preferred embodiment, the system employs a hybrid ensemble model composed of gradient boosting machines (GBMs), decision trees, and deep neural networks (DNNs) trained on historical fulfillment data, prescriber behavior, pharmacy performance records, and geographic access metrics. These models are continuously updated through online learning or periodic batch retraining pipelines using de-identified data, enabling them to adapt to evolving patterns in prescription demand, pharmacy responsiveness, and regional supply chain conditions.

Upon execution of a query, the AI engine retrieves relevant contextual parameters, including but not limited to: (i) the prescribing clinician's location and prescribing history; (ii) the patient's geographic coordinates or preferred pickup location; (iii) insurance formulary alignment; (iv) current and historical stock levels at candidate pharmacies; (v) average fill times and failure rates; and (vi) pharmacy operating hours and fulfillment capacity. These features are preprocessed, weighted, and input into the predictive model, which produces a ranked list of pharmacies optimized to fulfill the specified medication order in the shortest time with the highest confidence.

In certain embodiments, the system also factors in patient preferences explicitly collected through prior interactions or indirectly inferred through behavioral patterns. For example, if a patient consistently selects pharmacies affiliated with a particular retail chain or located near a specific transit route, the AI engine can prioritize those options in future recommendations. Additionally, the AI module may incorporate dynamic weighting schemes that adjust feature importance based on situational variables—such as favoring distance more heavily during off-peak hours or emphasizing fill reliability during periods of supply chain disruption.

The invention further supports real-time feedback loops that feed post-query outcomes (e.g., whether the prescription was successfully filled, delayed, or redirected) back into the AI training corpus. This feedback mechanism enhances the system's predictive accuracy and enables reinforcement learning capabilities, wherein the AI models iteratively refine their decision-making criteria based on the real-world success rates of their prior recommendations. These updates are performed in a privacy-preserving manner, with all feedback data anonymized and stripped of personally identifiable information (PII) prior to model ingestion.

The AI engine is encapsulated within a secure execution environment and exposed to upstream components via a dedicated AI inference API. This API receives contextual input, executes prediction logic, and returns ranked pharmacy recommendations along with associated confidence scores and rationale explanations. In one embodiment, the explanation layer is powered by SHAP (SHapley Additive exPlanations) or similar interpretability algorithms, allowing the system to surface to the end-user or system administrator the key factors influencing a given pharmacy recommendation.

In addition to real-time use cases, the AI subsystem supports batch analysis for population health optimization, wherein the system can analyze prescribing patterns across clinics or networks to identify geographic gaps in pharmacy accessibility, supply shortages, or non-optimized referral patterns. These insights may be surfaced to administrative stakeholders for operational planning, compliance reporting, or contract negotiations with pharmacy networks and payers.

The AI-powered pharmacy selection and fulfillment optimization engine enables the invention to move beyond simple availability queries and into the domain of intelligent decision support. By incorporating data-driven insights, adaptive learning algorithms, and explainable model outputs, the system delivers precision-guided pharmacy matching that enhances the likelihood of medication access, improves patient satisfaction, and supports more effective clinical workflows.

The present invention further comprises a real-time fulfillment feedback subsystem designed to capture, process, and transmit updates regarding the post-query status of prescription fulfillment events. This subsystem enables the RTRx platform to close the informational loop between the initial inventory query and the downstream dispensing activity, thereby enhancing clinical visibility, improving patient communication, and supporting dynamic prescription management. The fulfillment feedback mechanism is engineered to operate asynchronously and securely, leveraging bi-directional communication channels between the RTRx system and participating pharmacy management systems (PMSs).

In the preferred embodiment, upon successful submission of a prescription—whether electronically, by print, or through patient handoff—the system activates a passive fulfillment listener module. This module monitors for updates from the pharmacy regarding the status of the medication transaction, including but not limited to: (i) medication dispensed; (ii) medication out of stock; (iii) prescription placed on hold; (iv) partial fulfillment; and (v) prescription canceled or transferred. These updates are captured either through direct integration with the PMS, through standardized message protocols such as NCPDP SCRIPT status transactions, or through custom webhook implementations provided by the pharmacy.

The feedback subsystem utilizes an event-driven architecture, wherein each fulfillment update is treated as a discrete event published to a secure message queue or event bus. Events are tagged with metadata such as prescription ID, pharmacy ID, event timestamp, and status code. Upon publication, a fulfillment event handler consumes the message, performs schema validation and authorization checks, and routes the update to the appropriate upstream system, which may include the originating prescriber interface, patient portal, care coordination system, or EHR module.

The system includes logic for correlating fulfillment events with their originating inventory queries and prescription recommendations, enabling prescribers and clinical teams to assess the efficacy of pharmacy selection in real-world conditions. In embodiments where a selected pharmacy is unable to fulfill a prescription as initially indicated, the system may trigger a real-time re-query to suggest alternative fulfillment options or notify the prescriber of the disruption, thereby enabling proactive intervention. This feature is particularly critical for time-sensitive medications or in cases involving limited pharmacy access in rural or underserved areas.

To ensure compliance with data security and privacy regulations, all fulfillment events are processed in a de-identified manner, and patient-specific identifiers are only transmitted where expressly permitted under existing data-sharing agreements. The system employs end-to-end encryption and token-based access controls to secure fulfillment updates in transit and at rest. All transactions are logged in a tamper-evident audit trail for compliance, dispute resolution, and analytics purposes.

In certain configurations, the system supports proactive alerts and notifications, wherein users may opt-in to receive real-time updates via SMS, email, push notification, or in-application alerts based on their role (e.g., prescriber, care manager, patient). Alert thresholds and preferences may be configured at the user level or system-wide, enabling tailored workflows that improve medication adherence and transparency. For example, a clinician may receive a real-time notification if a high-priority prescription is not filled within a designated window, triggering follow-up action.

Additionally, the fulfillment feedback subsystem serves as a data source for performance analytics and pharmacy benchmarking. By aggregating fulfillment statuses across thousands of transactions, the system can generate pharmacy-level metrics such as average fill time, fulfillment reliability rate, and responsiveness to prescription activity. These metrics may be used to inform future AI-driven pharmacy recommendations, support payer and provider decision-making, or drive contractual negotiations with pharmacy networks.

The real-time fulfillment feedback and status update loop is a critical operational component of the invention, ensuring that inventory availability insights are coupled with downstream visibility into prescription outcomes. By integrating bidirectional communication with event-driven architecture, privacy-preserving mechanisms, and real-time alerting, the system enables a continuous feedback framework that improves medication accessibility, clinical coordination, and pharmacy network performance.

### Detailed Description of Figures

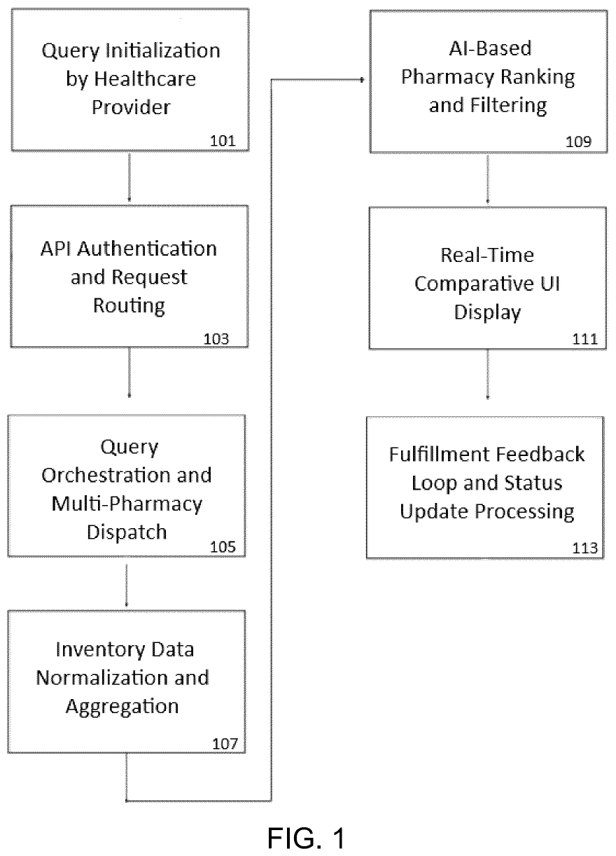

FIG. 1.101—Query Initialization by Healthcare Provider

A healthcare provider, using a clinical interface such as a web portal, EHR-integrated module, or mobile application, initiates a real-time medication availability query by specifying the desired medication and relevant contextual information such as patient location, prescription urgency, or preferred pharmacy networks. This input is structured into a digital query payload and transmitted to the RealTimeRx (RTRx) platform via a secure connection.

FIG. 1.103—API Authentication and Request Routing

Upon receipt of the query payload, the RTRx stateless API gateway performs authentication and access control verification, confirming the identity and authorization level of the requesting entity. Validated requests are then routed to the appropriate internal query orchestration module using predefined routing rules, sessionless logic, and audit tagging to maintain compliance with security and traceability standards.

FIG. 1.105—Query Orchestration and Multi-Pharmacy Dispatch

The query orchestration module interprets the incoming request and constructs customized outbound queries compatible with the communication protocols of one or more pharmacy management systems (PMSs). In multi-pharmacy mode, the module concurrently dispatches inventory requests to a network of pharmacies, using adapter interfaces or middleware agents to accommodate both standardized and proprietary pharmacy systems.

FIG. 1.107—Inventory Data Normalization and Aggregation

As responses are received from PMS endpoints, the interoperability layer standardizes the disparate data formats into a unified schema through transformation logic, field mapping, and pharmaceutical code normalization. This aggregation engine consolidates inventory data into a cohesive structure that facilitates consistent comparative analysis and downstream presentation.

FIG. 1.109—AI-Based Pharmacy Ranking and Filtering

An embedded artificial intelligence (AI) engine ingests the normalized inventory dataset and applies predictive models to rank pharmacy options based on predefined and dynamic criteria, including but not limited to medication availability, geographic proximity, patient insurance compatibility, historical pharmacy performance, and expected fulfillment timelines. The output is a prioritized list of pharmacies optimized for fulfillment success.

FIG. 1.111—Real-Time Comparative UI Display

The ranked inventory data is rendered on a user interface in real-time, using interactive visual elements that may include pharmacy names, availability indicators, distance metrics, stock confidence scores, and estimated fill times. The interface allows for filtering, sorting, and direct selection, and is compatible across clinical web platforms, EHRs, and mobile applications.

FIG. 1.113—Fulfillment Feedback Loop and Status Update Processing

After a prescription is issued, the system monitors fulfillment events through webhook listeners, standard messaging protocols, or agent-based polling. Status updates such as filled, delayed, or out-of-stock are received from the PMS and pushed back to the provider interface. This feedback enables real-time intervention, automatic re-querying, or patient notification workflows, thereby completing the inventory-to-fulfillment lifecycle.

What is claimed is:

1. A system for real-time retrieval and presentation of pharmacy inventory availability, comprising:
a. a processor;
b. a memory coupled to the processor;
c. a stateless application programming interface (API) configured to receive a real-time inventory query initiated by a healthcare provider device, the query specifying a medication and a patient context;
d. a query orchestration module in communication with the API, configured to construct and transmit at least one outbound request to a plurality of pharmacy management systems (PMSs), each request formatted in accordance with a protocol associated with the corresponding PMS;
e. an interoperability layer configured to normalize and standardize responses received from the plurality of PMSs into a unified data schema;
f. a front-end interface rendering engine configured to generate a real-time comparative display of medication availability across multiple pharmacies, wherein the display includes availability indicators, geographic information, and pharmacy identifiers;
g. an artificial intelligence (AI) module configured to rank the plurality of pharmacies based on a weighted set of criteria comprising inventory level, geographic proximity to a patient, pharmacy affiliation, insurance compatibility, and historical fulfillment performance;
h. and a fulfillment feedback subsystem configured to receive, process, and display status updates from one or more PMSs concerning the downstream prescription event, including at least one of: dispensed, delayed, canceled, or out-of-stock, wherein the system is configured to execute each query and return availability data to the healthcare provider device without persisting inventory data in memory or storage beyond the life of the transaction.

2. The system of claim 1, wherein the AI module comprises a machine learning model trained on historical prescriber behavior, pharmacy fulfillment records, and patient location data.

3. The system of claim 1, wherein the interoperability layer includes adapter modules configured to translate between proprietary PMS protocols and the unified data schema.

4. The system of claim 1, wherein the front-end interface comprises a user interface embedded in an electronic health record (EHR) platform using a SMART on FHIR-compatible module.

5. The system of claim 1, wherein the fulfillment feedback subsystem uses webhook listeners or SCRIPT-standard messaging protocols to receive asynchronous status updates.

6. A method for querying and presenting real-time pharmacy inventory information without persistent data storage, the method comprising:
a. receiving, via a stateless application programming interface (API), a real-time inventory availability query from a healthcare provider device, the query including a medication identifier and patient-related data;
b. routing the query to a query orchestration module configured to format and transmit outbound requests to at least one pharmacy management system (PMS);
c. receiving inventory availability responses from the at least one PMS;
d. normalizing the inventory responses into a unified schema through an interoperability layer;
e. rendering, via a user interface, a real-time comparative visualization of inventory availability across a plurality of pharmacies;
f. executing, via an artificial intelligence (AI) engine, a ranking operation on the plurality of pharmacies using input parameters comprising inventory status, patient proximity, insurance compatibility, pharmacy reliability, and prescriber history;
g. and receiving, via a fulfillment feedback module, post-query prescription fulfillment status updates from at least one PMS,
wherein the method executes each inventory query and returns data to the user interface without storing the inventory data in a persistent memory or storage system.

7. The method of claim 6, wherein the AI engine updates its ranking logic over time using reinforcement learning based on prescription fulfillment outcomes.

8. The method of claim 6, further comprising filtering displayed pharmacy options based on formulary constraints, patient preferences, or network affiliation policies.

9. The method of claim 6, wherein the user interface is accessible via at least one of: a web browser, mobile application, or an embedded EHR component.

10. The method of claim 6, wherein fulfillment status updates are used to automatically re-query alternative pharmacies in response to fulfillment failure.
